(19) United States
(12) Patent Application Publication
(10) Pub. No.: US 2024/0100278 A1
(43) Pub. Date: Mar. 28, 2024

(54) ENDOTRACHEAL TUBE

(71) Applicant: THE BOARD OF REGENTS OF THE UNIVERSITY OF TEXAS SY, Austin, TX (US)

(72) Inventors: DAVID BERARD, San Antonio, TX (US); ISAAC TREVINO, San Antonio, TX (US); YUSHENG FENG, San Antonio, TX (US); ROBERT L. HOOD, San Antonio, TX (US); ROBERT DELORENZO, San Antonio, TX (US); DAVID RESTREPO, San Antonio, TX (US)

(21) Appl. No.: 17/769,181

(22) PCT Filed: Oct. 16, 2020
(86) PCT No.: PCT/US2020/056090

§ 371 Date: Apr. 14, 2022

Related U.S. Application Data
(60) Provisional application No. 62/916,011, filed on Oct. 16, 2019.

(51) Int. Cl. A61M 16/04

(52) U.S. Cl. CPC A61M 16/0436; A61M 2205/0216

(57) ABSTRACT

Certain embodiments include an endotracheal (ET) device assembly including an expansion lattice coupled with an expansion and/or contraction actuator. In certain aspects the expansion lattice is position within an open-ended sheath or cover that is position between the trachea and the expansion lattice when deployed.

### PRIORITY PARAGRAPH

This Application claims priority to U.S. Provisional Patent Application 62/916,011 filed Oct. 16, 2019, which is incorporated herein by reference in its entirety.

### STATEMENT REGARDING FEDERALLY FUNDED RESEARCH

None.

### BACKGROUND

Current practices for airway management in traumatic injury care are profoundly inadequate. During immediate care of a trauma patient, securing the airway is often the top priority for the attending provider. This is true regardless of the source of the airway obstruction—whether it be face and neck trauma from gunshot or knife wounds, or from an automobile accident. Compromised airways are among the most frequently encountered injuries in emergency medical settings. Tracheal intubation errors occurred in 23% of attempts in one pre-hospital civilian study. Advanced techniques such as the use of anesthetics or muscle relaxants appear to improve the chances of success, but these methods require extensive training and practice to use safely. Surgical airways such as cricothyrotomy are often promoted as an alternative, but pre-hospital failure rates ranging from 18-33% suggest this approach is not a viable solution.

Discussions with experienced medics and emergency health professionals have identified possible shortcomings, outside of training and experience limitations, in the standard-of-care approach to endotracheal intubation: (1) Failure to locate the trachea because of injury, swelling, presence of debris, etc.; (2) Inability to insert the ET tube properly because of environmental conditions and trauma, and because the tube is a large, fixed-diameter, inflexible device that does not adapt well to anatomical variations; and (3) Improper securement resulting in unintended dislodgement, leading to airway failure.

There remains a need for additional endotracheal tubes having components that address a variety of the problems described above.

### SUMMARY

To address problems with current devices and methods a re-design/re-engineering of the endotracheal (ET) tube was pursued. A solution to some of the problems associated with ET tube includes: (a) Providing a design that distributes pressure across a larger cylindrical shaped contact area when the ET tube is deployed and anchored, which lowers the maximum pressure needed to form a better seal. (b) Utilizing a physical actuation mechanism in place of pneumatic actuation, which results in the decoupling of the expansion mechanism from environmental conditions/influence, e.g., changing air pressure. (c) Providing for integrating of various sensors to detect proper placement and pressure levels etc., which lowers the risk of immediate and downstream patient complication by simplifying use and operation. Certain embodiments include an ET device assembly having a proximal end that remains outside of the trachea and a distal end that inserted into the trachea. The distal portion of the device includes an ET tube portion and an expansion lattice circumscribing the ET tube. The lattice when contracted along the long axis secures the ET tube once deployed, i.e., the expansion lattice is expanded along the long axis during deployment and contracted to secure its placement. The ET device assembly can include an expansion lattice coupled with an expansion and/or contraction actuator. In certain aspects the expansion lattice is positioned within an open ended sheath or cover (protection membrane) that is position between the trachea and the expansion lattice when deployed. In other aspects, the ET device includes a conduit along at least a portion of the device to provide a path for wires and other connections for sensors and the like.

Performance of the device, and in particular the expansion lattice, can be characterized regarding expansion time, radial forces, and reversible expansion and contraction. The device can be configured to ensure sensor integration does not impede or hinder mechanical actuation. Other characteristics are that there is minimal device motion once the device is secured. In certain embodiments the device reduces tissue damage (abrasion, hoop stress, etc.) relative to the current clinical standard-of-care, e.g., balloon inflation.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-F. Illustrates one embodiment of an ET device and various aspects of the expansion lattice. (FIG. 1A), illustration of one embodiment of a ET device assembly. (FIG. 1i), contracted cylindrical lattice. (FIG. 1C), extended cylindrical lattice. (FIG. 1D), illustration of one embodiment of expansion lattice components. (FIG. 1E), illustration of one example of initiation of expansion lattice assembly. (FIG. 1F), illustration of expansion lattice operation.

FIGS. 2A-B. Illustration of two embodiments of a joint that can connect strips and provide a pivot point.

FIG. 3. Illustration of a second embodiment of a joint mechanism that can connect strips and provide a pivot point.

FIG. 4. An illustration demonstrating the physical and mathematical basis analytical expressions related to describing the cylindrical lattice.

FIG. 5. Illustration comparing experimental to theoretical diameter and length in two separate embodiments.

FIGS. 6A-6B. Airway Securement and Integrated Clearance System (AirSINC). (A) Parts description. (B) AirSINC with architected lattice in expanded configuration and extended suction hose.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Technological improvements in airway management devices have not kept pace with hemorrhage control, wound stabilization, and other methods of preventing and/or containing life threatening, traumatic injuries. To address this fact, re-design/re-engineering of ET tubes was undertaken. Certain aspects of the re-design can improve ET tube placement and securement, resulting in fewer airway failures, better pre-hospital care, and fewer airway-related deaths from trauma casualties. The advances described can benefit research and medical practice related to emergency medicine, first responders, surgical procedures, burn care, infection prevention and much more. The re-design/re-engineering results in an airway device (ET device) that ensures mechanical securement of the device to the patient to reduce the overall failure rate.

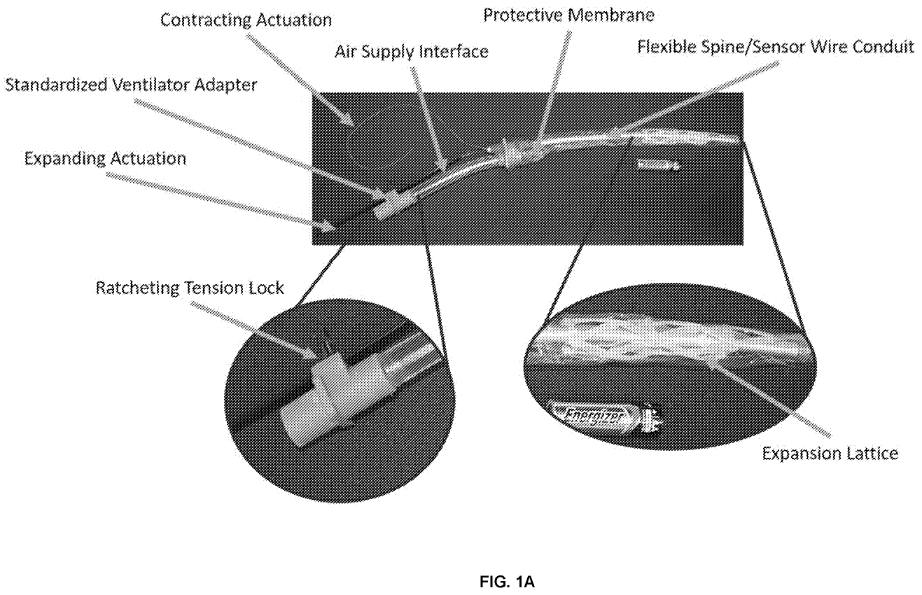

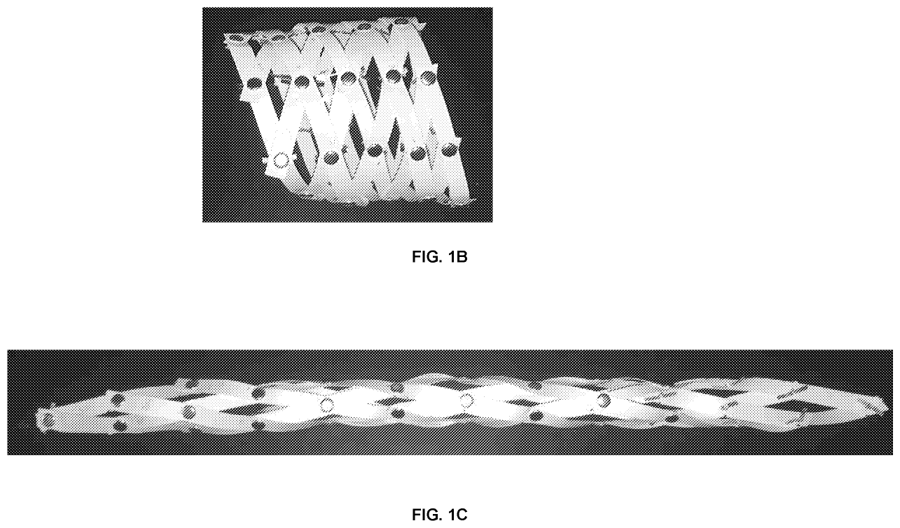

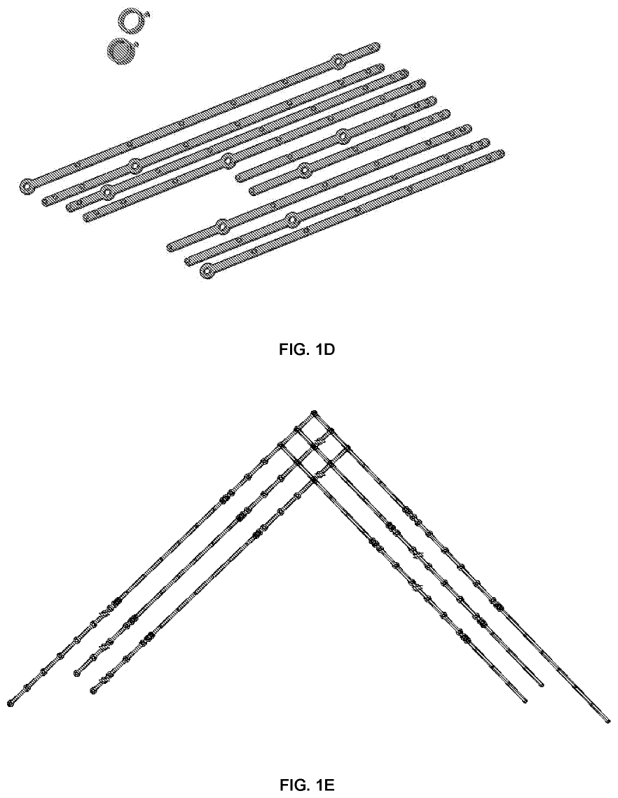

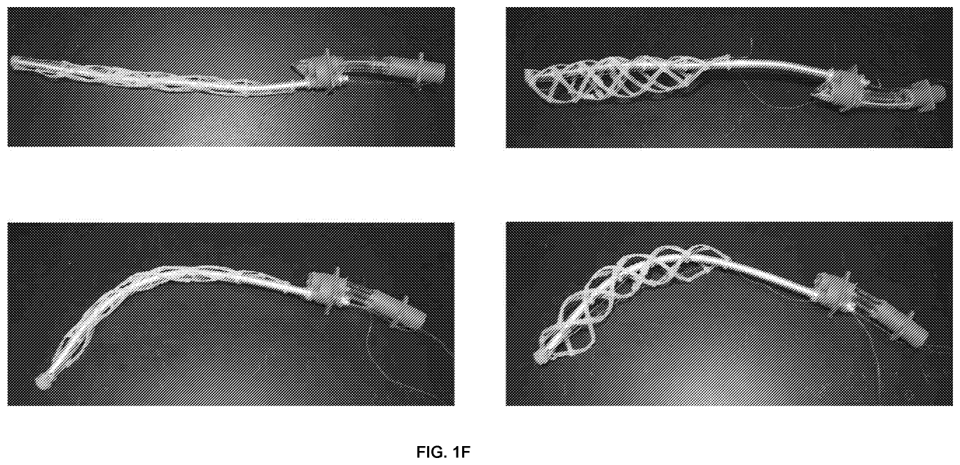

FIG. 1A provides an illustration of one embodiment of an ET device assembly. An ET device is shown having a proximal end or portion that is configured to remain outside of the patient and a distal end or portion that is to be inserted into the trachea of a patient. The device includes a proximal ventilator adapter coupled to an air supply interface. The air supply interface is further coupled to the distal ET tube portion. The ET tube portion is circumscribed or position inside an expansion lattice portion, the expansion lattice portion being a cylindrical lattice. The expansion lattice portion is optionally contained within or covered by a protective membrane (sheath or cover), that upon deployment of the device the protective membrane can be positioned between the patient and the expansion lattice. The sheath or cover being tubular in shape, affixed to the proximal portion of the distal portion, and having a distal opening providing the ET tube access to the lumen of the trachea. The expansion lattice can be coupled to one or more actuators that expand and/or contract the lattice as needed. Expansion of the lattice reduces its diameter and allows the insertion of the ET device. Once positioned, the lattice is contracted, expanding its diameter and securing the ET device in the patient. In certain aspects, a conduit is included along the long axis of the device providing access (wires etc.) to a variety of sensors that can be positioned along the device at various positions and orientations. FIG. 1B illustrates a representation of an expansion lattice in a contracted configuration and FIG. 1C illustrates a representation of an expansion lattice in an expanded configuration.

In certain embodiments an ET device assembly will include an expansion lattice, a cylindrical lattice. The cylindrical lattice is produced from strips (FIG. 1D shows one embodiment of the strip component) woven into a lattice with the strips being connected by joints that provide pivot points between two strips. FIG. 1E shows the initiation of weaving process with six strips. The number of strips can vary from 2, 3, 4, 5, 6, 7, 8, 9, to 10 or more strips. The lattice can be operatively coupled to an actuation mechanism. The actuation mechanism can be a single point actuation mechanism. The actuation mechanism will provide or allow for the contraction of a lattice once it is positioned appropriately, which will secure the ET device in position. The actuation mechanism can also provide for an expansion of a deployed lattice to allow for removal of the ET device. FIG. 1F illustrates the operation of one embodiment of an ET device as described herein, with the left hand illustrations being an expanded configuration and the right hand illustrations of a contracted configuration. In certain aspects the expanded inner diameter of the lattice is 0.5, 0.75, 1, 5, 10, 15, to 20 mm, and a contracted outer dimeter of 0.5, 1, 5, 10, 15, 20, 25, 30, 35, 40, 45, to 50 mm or larger.

The term “strip” is representative. A strip of the device is an elongated component having a length, width, and thickness. The strip forming an interior face that faces the central axis of the cylindrical lattice and an external face that faces the exterior of the cylindrical lattice. The strip can have a variety of cross-sectional shapes, e.g., circular (as in wires), elliptical, rectangular, or any other polygonal cross-section. The edges of the strips can be beveled, rounded, or squared. A strip can have a length of 5, 10, 15, 20, 25, 30, 40, 50, 60 mm or more, including all values and ranges there between. In certain aspects, the strip are between 5 and 20 mm in length. In certain aspects, the strip can have width of 100, 200, 300, 400, 500, 600, 700, 800, 900 μm to 1, 2, 3, 4, 5, 6, 7, 8, 9, 10 mm or larger, including all values and ranges there between. In certain aspects, the strip can have a thickness of 10, 25, 50, 100, 200, 300, 400, 500, 600, 700, 800, 900 μm to 1, 2, 3, 4, 5 mm, including all values and ranges there between. The strips forming a lattice can the same, similar (+/−5%) or different dimensions.

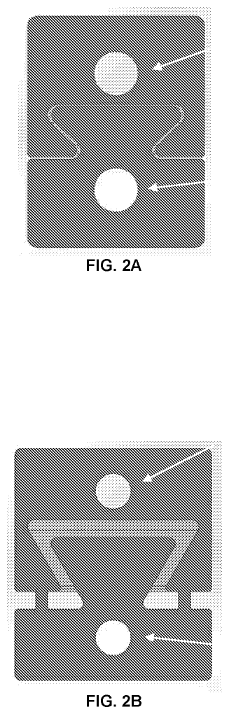

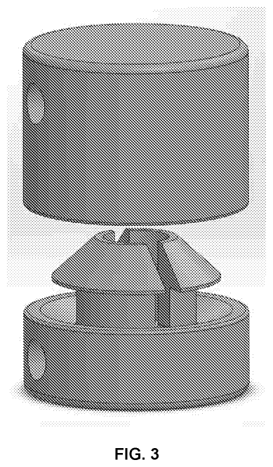

The strips of a lattice are joined together at specified points along their length in a helical pattern—the points of attachment forming pivot points. The strips can be joined by any mechanism that limits their movement to one degree of freedom, rotation about the axis normal to the intersection of the strips. Some examples of this are a mechanism that is part of the geometry of the strip, fasteners, joints, hinges, or compliant mechanisms. FIG. 2 and FIG. 3 provide non-limiting examples of joint mechanisms that be employed. In certain aspects, the acceptor and/or donor portion of a joint can be fabricated as a part of the strip. Each strip can have 0, 1, 2, 3, 4, 5, 6, 7, 8, 9, 10, 11, 12, 13, 14, 15, 16, 17, 18, 19, 20 or more joint acceptor or join donor portions that can be assembled to form a joint. In other aspects each strip can have 0, 4, 5, 6, 7, 8, 9, 10, 11, 12, 13, 14, 15, 16, 17, 18, 19, 20 or more holes for insertion of an attachment mechanism. In still other embodiments, the attachment mechanism forms a lumen through which a strip is positioned, e.g., the strip need have a hole to accommodate the attachment mechanism if the attachment mechanism has a lumen to accommodate the strip.

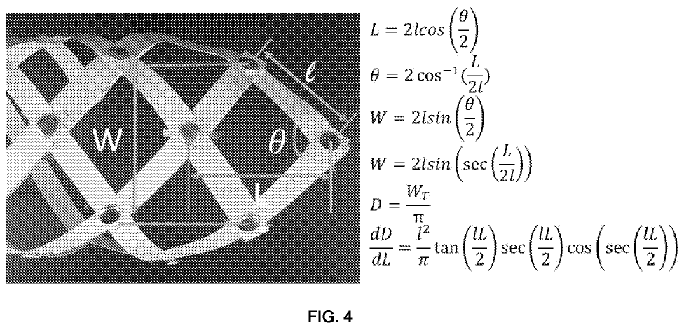

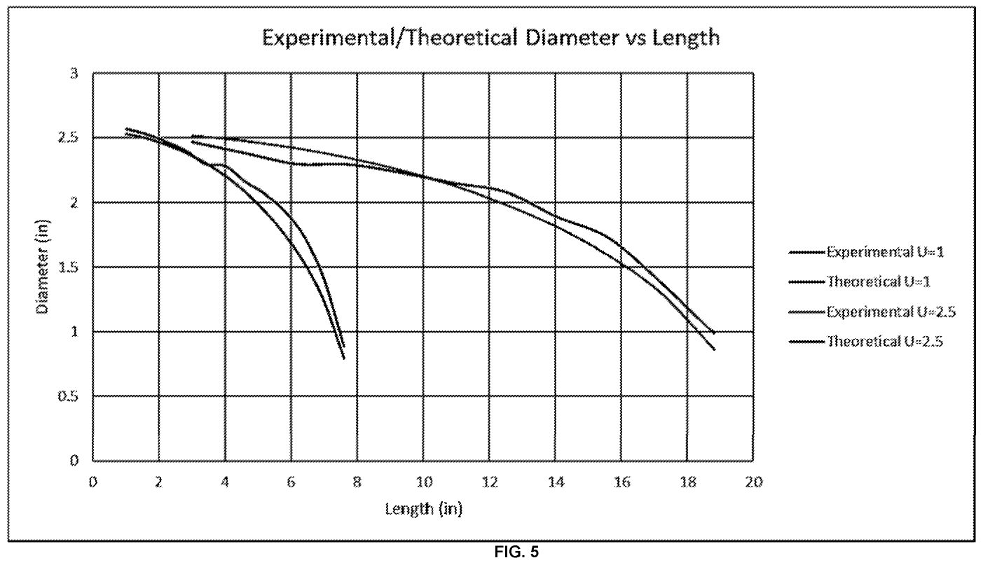

The lattice can have a minimum of 2 strips and can be constructed with an even or odd number of total strips. In certain aspects, at least one of the strips must coil in the opposite direction (clockwise or counter-clockwise) to the others. When assembled, strips that coil in the same direction may be oriented to be parallel with respect to each other or not. The ratio of strips coiled in opposite directions will affect the expansion characteristics and can be chosen depending on the intended use. Ratios closer to 1 will yield a more evenly expanding/contracting cylinder. Ratios farther away from 1 (1:6, 1:5, 1:4, 1:3, 1:2, 2:1, 3:1, 4:1, 5:1, 6:1) can induce uneven expansion/contraction that may be favorable in some applications. The behavior of the cylinder is determined by the number of strips used and the distance between the attachment points. In certain aspects the attachment points can be evenly spaced or unevenly spaced. A greater number of strips will increase both the minimum diameter of contraction and the maximum diameter of expansion. A larger distance between the joints will increase the maximum diameter of expansion but will not affect the minimum diameter of contraction. The distance between adjacent attachment points along a strip can be 1, 2, 3, 4, 5, 6, 7, 8, 9, 10, 15, 20, 25, 30, 35, 40, 45, 50 mm or longer, including all values and ranges there between, as measured from the center of the attachment points. Both a greater number of strips and a larger distance between attachments produce a greater rate of radial expansion with respect to the change in axial length. The distance between attachment points does not need to be equal throughout. Varying distances between attachments will produce a cylinder with a variable diameter along its length. A larger number of strips and shorter distance between attachment points will yield a denser lattice cylinder and can significantly increase maximum expansion force exerted and radial stability while maintaining bending compliance. Strips can be made to be modular so that sections of a lattice cylinder of a certain length can be combined together enabling longer cylinders that still actuate as a single body. FIG. 4 illustrates a number of mathematical determinations for characterization of the expansion lattice. FIG. 5 illustrates the experimental/theoretical diameter versus length of one example of such a lattice.

The type of material used to make the strips will affect the mechanical characteristics of the lattice. A stiffer material will enable a greater amount of expansion pressure. Strips can be made from a variety of materials, including but not limited to metals, plastics, and combinations thereof. Plastics can include material such as polyvinyl chloride (PVC), polypropylene, polyethylene, polystyrene, polyethylene terephthalate (PET), polyimide, polycarbonate (PC), acrylonitrile butadiene, polyether ether ketone (PEEK), polyurethane, and ultra-high molecular weight polyethylene (UHMWPE). Metal materials include metals and metal alloys such as stainless steel, nickel-titanium, and the like. Other materials can include wood, bamboo, other organic compounds with flexibility. The strips can be formed by molding, printing, stamping, or the like.

The Covid-19 crisis has exposed several shortcomings in the current standard of care for severely ill patients, particularly those requiring prolonged ventilation and tracheal intubation (Chavez, et al., The American journal of emergency medicine, 2020: p. S0735-6757(20)30178-9): (1) Prolonged intubation can lead to tracheal stenosis at the cuff site, ulceration, dislocation, or scarring and stricture of the arytenoid cartilages. Such injuries are particularly prone to occur if an oversized endotracheal tube or over-pressurized cuff is used or is left in position for longer than a week (Cooper, Thorac Surg Clin, 2018. 28(2):139-144); (2) Traditional polyvinylchloride tracheal tubes with low pressure cuffs are prone to device failure by means of cuff failure, poor seating, and tube cracking, all of which may cause leaks with attendant loss of ventilation efficacy and, importantly, the risk of virally-contaminated air escaping into the local atmosphere (Paramaswamy, Ain-Shams Journal of Anesthesiology, 2019. 11:1-4). Traditional tubes are also prone to dislodgement, with as many as 3% of prehospital intubations suffering this adverse event (Wang, et al., Resuscitation, 2009. 80(1):50-5), and 11-13% becoming dislodged in the hospital setting (Carrión et al., Crit Care Med, 2000. 28(1):63-6); (3) The process of intubation requires close patient contact exposing healthcare providers (HCPs) to aerosolized secretions elevating the risk of virus transmission (Meng et al., Anesthesiology, 2020. 132(6):1317-1332; Chen and Zhao, The Journal of hospital infection, 2020. 105(1):98-99; Vesoulis and Edwards, TIME Magazine, 2020. 195(12/13):32-33). Exacerbating this situation are shortages of personal protective equipment, as well as overwhelmed EMS systems, EDs and ICUs that cannot provide for optimal environmental controls such as negative pressure rooms (Chen and Zhao, The Journal of hospital infection, 2020. 105(1):98-99; Vesoulis and Edwards, TIME Magazine, 2020. 195(12/13):32-33). Since HCP risk is likely dose- and duration-dependent, a device that maximizes intubation success and minimizes procedure time will significantly mitigate virus transmission (Cook, Anaesthesia, 2020. 75(7):920-927). (4) Intubation is a complex manual skill requiring considerable hand-eye coordination (Tarasi et al., Medical education online, 2011. 16:10.3402/meo.v16i0.7309). Successful intubation requires the nearly simultaneous manipulation of as many as four implements: endotracheal tube, bougie/stylet, laryngoscope, and suction catheter while also controlling patient head and neck position. These four implements must be skillfully and separately guided within the tight confines of the oropharynx and changed out as the procedure progresses or evolves. The minimum result is delayed intubation as multiple hand and eye movements are required as each implement is used and subsequently exchanged, while the worst outcome is psychomotor confusion and failed intubation. Integrating the key functions of bougie or stylet, and endotracheal tube into a single, smoothly operating, multi-functional unit will likely decrease the time to ventilation and increase first pass success rates, both key outcome indicators in airway management.

New developments in ETTs have the potential of producing a paradigm-shifting impact to the standard of care in airway management. Currently, a chief obstacle to airway management is the standard polyvinylchloride ventilation tube itself: a large, inflexible, fixed-diameter tube that must navigate a sinuous airway and traverse a relatively small glottic opening that varies by patient size, age, and condition. This tube is made from a relatively inexpensive, easily mass-produced material that can have substantial material property variation under the dynamic ambient conditions of the battlefield. Further complications arise when maxillofacial injuries are present, as locating and navigating the airway can be difficult or even impossible with current equipment. The design has seen minimal improvements and research attention over decades and is a major contributor to the deficit in casualty airway management currently observed in the field.

Embodiments described herein address some of the problems described above and provide an Airway Securement and Integrated Clearance System (AirSINC). Some of the advantages of the devices described herein include: (1) Radially expanding endotracheal tube that also functions as a narrow bougie for easier insertion, and contracts for removal. (2) Intubation tube with distributed securement area to avoid tube dislodgment, micro-aspiration, and tissue damage due to cuff pressure. (3) Limiting exposure of healthcare providers to patient pathogens through streamlining the intubation process and facilitating suction to maintain airway clearance and evacuate aerosols. (4) Robust and easy-to-use airway management device that minimally trained operators can use to clear and secure the airway. (5) Eliminating the need of multiple size intubations tubes: one tube fits all; and integrated bougie and oropharyngeal/tracheal catheter functions reduces necessary equipment to carry.

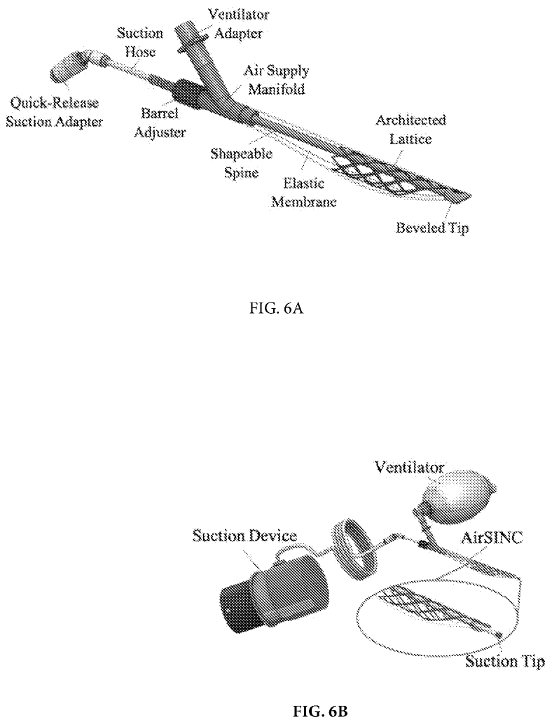

A schematic of one embodiment of a device (e.g., AirSINC) is shown in FIG. 6. FIG. 6 shows the system integrated with a respirator and suction device. A description for AirSINC is as follows: referring to FIG. 6A, at the proximal end of there is a universal adapter that allows any suction device to be attached to the suction hose. Prior to cannulation and securement of the trachea, the distal tip of the device can be used as an oropharyngeal suction catheter. The tip diameter can be manipulated at need with the architected lattice at need if larger solid phase materials are present. The suction hose has a threaded section at the interface termed the Barrel Adjuster (see FIG. 6B). This mechanism can be rotated to extend a small suction catheter from the distal end of the device for bronchial suction once the airway is secured. The air supply manifold serves as the interface for the suction and ventilation sources. The first inlet attaches to the barrel adjuster, and the second has a standardized 15 mm adapter that attaches to ventilation. The outlet is attached to an elastic membrane, which creates a sealed environment from the ventilation source to the patient. Near the distal end of the AirSINC, there is an expanding architected lattice. This lattice replaces the high-volume low-pressure standard securement cuff in current intubation tubes. The lattice enables a larger and more even distribution of the contact forces, which can reduce injuries and tissue damage commonly associated with current endotracheal tubes. The lattice also permits the device to start at a smaller diameter than most adult tubes and expand to the largest adult sizes, essentially performing as a one-size-fits-all device. This feature will enable standardized placement and expansion procedures and reduce incidence of incorrectly sized tubes and associated complications. The open configuration of the lattice is shown in FIG. 6B.

1. An endotracheal (ET) device assembly comprising an expandable lattice positioned around at a portion of the endotracheal tube circumference, the lattice comprising a plurality of longitudinal strips connected at a plurality of pivot points forming an expandable and retractable cylindrical lattice.

2. The assembly of claim 1, wherein a first longitudinal strip and a second longitudinal strip are connected by a fastener passing through coincident holes formed in the first longitudinal strip and second longitudinal strip.

3. The assembly of claim 1, wherein a first longitudinal strip forms a receiving portion of a coupling mechanism and a second longitudinal strip forms a donating portion wherein the receiving portion complements the donating portion and interacts to form a pivot point between the first longitudinal strip and the second longitudinal strip.

4. The assembly of claim 1, wherein the distance between two consecutive attachment points on a strip is 1/32, 1/16, 3/32, ⅛, 5/32, 3/16, 7/32, or ¼ of an inch.

5. The assembly of claim 1, wherein the cylindrical lattice is expanded or contracted by a fixed point actuator operatively coupled to the lattice.

6. An expandable lattice comprising a plurality of longitudinal strips connected at a plurality of pivot points forming an expandable and retractable cylindrical lattice.

7. The lattice of claim 6, wherein a first longitudinal strip and a second longitudinal strip are connected by a fastener passing through coincident holes formed in the first longitudinal strip and second longitudinal strip.

8. The lattice of claim 6, wherein a first longitudinal strip forms a receiving portion of a coupling mechanism and a second longitudinal strip forms a donating portion wherein the receiving portion complements the donating portion and interacts to form a pivot point between the first longitudinal strip and the second longitudinal strip.

9. The lattice of claim 6, wherein the distance between two consecutive attachment points on a strip is 1/32, 1/16, 3/32, ⅛, 5/32, 3/16, 7/32, or ¼ of an inch.

10. The lattice of claim 6, further comprising a fixed point actuator operatively coupled to the lattice for expansion and/or contraction of the lattice.
